(19) United States
(12) Patent Application Publication
(10) Pub. No.: US 2004/0229821 A1
(43) Pub. Date: Nov. 18, 2004

(54) Orally administrable pharmaceutical compositions and methods for preventing food-drug interaction

(76) Inventors: Ae-Ri Kim, Yuseong-gu (KR); Kwan-Hyung Cho, Yuseong-gu (KR); Ji-Hye Lee, Yuseong-gu (KR); Sun-Hwa Lee, Yuseong-gu (KR); Ho-Jun Kim, Yuseong-gu (KR); Han-Joo Maeng, Yuseong-gu (KR); Hee-Dong Park, Yuseong-gu (KR); Tae-Hun Kim, Yuseong-gu (KR); Bong-Chan Kim, Yuseong-gu (KR); Sung-Ji Kim, Yuseong-gu (KR); Kyo-Seok Choo, Yuseong-gu (KR)

Correspondence Address: BIRCH STEWART KOLASCH & BIRCH, PO BOX 747, FALLS CHURCH, VA, 22040-0747, US

(21) Appl. No.: 10/782,951

(22) Filed: Feb. 23, 2004

(30) Foreign Application Priority Data
Feb. 24, 2003 (KR) 10-2003-0011392

(51) Int. Cl. A61K 38/04

(52) U.S. Cl. 514/19; 514/210.17

(57) ABSTRACT

The present invention provides an orally administrable pharmaceutical composition for preventing the decrease in the bioavailability of a drug after food intake, comprising, i) said drug; and ii) a pharmaceutically acceptable bioadhesive polymer, particularly for effectively preventing the decrease in the drug absorption rate due to interaction with digestive enzymes or food ingredients after food intake.

### TECHNICAL FIELD

The present invention relates to orally administrable pharmaceutical compositions and methods for preventing ‘food effect’ or ‘food-drug interaction,’ particularly, decrease in the drug absorption rate upon administering the drug after food intake. More specifically, the present invention relates to the pharmaceutical compositions and the methods for preventing decrease of the drug absorption rate in case the orally administered drug interacts with digestive enzymes secreted from the gastrointestinal tract or food ingredients after food intake.

### BACKGROUND ART

Bioavailability of oral formulations may vary due to the drugs' own properties, e.g. dissolution rate, absorption rate, and first-pass effect of the drugs, differences between individuals, e.g. in absorption, metabolism, or excretion rate of the drugs, and interaction of the drugs with other concurrently administered drug (drug-drug interaction) or food (food-drug interaction).

‘Food effect’ is bioavailability change (increase or decrease) of a drug induced by food intake. A side effect or toxicity may be a concern when bioavailability is increased after food intake. By contrast, when bioavailability is decreased after food intake, the blood concentration of the drug may not meet the effective concentration, and thus, the desired pharmacological effect may not be obtained (B. N. Singh, Effects of food on clinical pharmacokinetics, Clinical Pharmacokinetics 1999 37: 3, 213-255; I. Gauthier and M. Malone, Drug-food interactions in hospitalized patients, Drug Safety 1998 18: 6, 383-393). In particular, such food effect is very important for a drug whose blood concentration must be controlled within a narrow range to express its pharmacological effect without any side effect due to its narrow therapeutic window. Thus, in such case, patients' non-compliance may result in very serious problems. Therefore, during the development of all pharmaceutical products, the effect of food intake on a drug's absorption should be tested, and with such test results, it can be determined whether the drug may be administered regardless of food intake, or should be administered before or after food intake.

A physicochemical interaction between food and drug is one of the mechanisms to explain the food-drug interaction. For instance, when a drug's solubility largely depends on the pH of the solution, its dissolution rate is changed depending on the change of pH in the gastrointestinal tract by food intake, thereby changing the drug absorption rate. Its well-known example is an AIDS therapeutic, indinavir. This is a weak basic drug, and its dissolution and absorption rates are decreased when the pH of the stomach is raised by food intake, and thus, it should be taken one hour before or two hours after food intake. Further, a drug may form an insoluble complex with a metal ion present in food to result in the decrease of its absorption rate, whose examples are tetracycline and antibiotics of fluoroquinolone series. In addition, such drugs as alendronic acid, ciprofloxacin, clodronic acid, didanosine, digoxin, doxycycline, etidronic acid, norfloxacin, penicillamine, phenytoin, etc. were known to have a decreased absorption rate because the drugs are chelated or interact with food ingredients, for example, metals, fibers, etc. [. E. Schmidt and D. Kim 2002 Drugs 62(10) 1481-1502]. On the other hand, some lipophilic drugs with extremely low solubility in water have an increased dissolution time due to the delaying of gastric emptying by food intake or an increased absorption due to the dissolution by bile juice secreted after food intake.

Another mechanism of food-drug interaction is a metabolic interaction which food affects the metabolism of a drug to change its bioavailability. For example, bioavailability of cyclosporine or ketoconazole is increased by its reduced metabolism when grapefruit juice is taken.

Thus, identifying the food-drug interaction mechanism can lead to the development of its formulation that can improve the food-drug interaction. For example, when the food-drug interaction results from decrease in the solubility of a drug, the food effect might be minimized by designing a formulation to increase the solubility of the drug. Also, since the crystalline structure of a drug affects its dissolution rate, there has been an example to improve the change in the drug absorption rate by modifying the crystalline structure (U.S. Pat. No. 5,294,615). However, when a drug is absorbed only at a particular site of the gastrointestinal tract, particularly, the upper gastrointestinal tract, or when a drug has a low permeation rate across the membrane despite high solubility in water, its absorption rate may be decreased by food intake. In such a case, the food-drug effect can hardly be improved by designing new formulations, and its membrane permeation rate Should be improved by modifying the drug molecule itself [Pao et al., Reduced systemic availability of an antiarrhythmic drug, bidisomide, with meal co-administration: relationship with region-dependent intestinal absorption. Pharm. Res. 1998: 15(2) 221-227].

Various mechanisms were known for food-drug interaction, but such food-drug interaction cannot be inferred from the chemical structure or series of a drug (B. N. Singh, Effects of food on clinical pharmacokinetics, Clinical Pharmacokinetics 1999 37: 3, 213-255). On the other hand, U.S. Pat. Nos. 6,338,857 and 6,368,628 reported a composition to improve the change in bioavailability by food intake, but could not identify its mechanism, and further the composition was to improve the bioavailability increase by food intake, not to apply to the bioavailability decrease.

In the U.S. Ser. No. 10/280,587, the present inventors first disclosed an interaction between food and digestive enzymes as the mechanisms of food-drug interaction, which had not been previously known. According to the known physiology of the digestive system, the secretion of secretin and cholecystokinin (CCK), digestive hormones, is activated after food intake (V. S. Luciano, Human Physiology 5^{th} Ed. Chap 16 The digestion and absorption of food). Secretin activates the secretion of HCO₃⁻ to neutralize acids flowing from the stomach to the small intestine. Amino acids or fatty acids in the small intestine activate CCK and stimulate the secretion of various digestive enzymes secreted from the pancreas. Such digestive enzymes include trypsin, chymotrypsin, carboxypeptidase, lipase, amylase, ribonuclease, deoxyribonuclease, etc. These enzymes are involved in the digestion of proteins, lipids, carbohydrates, and nucleic acids. These digestive enzymes are secreted in the inactivated form and activated by other enzymes in the duodenum. Trypsin, particularly among the digestive enzymes, plays an important role as a digestive enzyme for proteins taken from food, and further, is involved in the activation of other inactive digestive enzymes. After food intake, trypsin is increased in the small intestine, and if a drug with an activity on trypsin is administered at this point of time, the drug interacts with trypsin and so the drug absorption is inhibited, and therefore, its bioavailability becomes lower than when it is administered on an empty stomach.

In the pharmacokinetic test using dogs and rats, one of the thrombin inhibitors disclosed in WO 00/39124, (2S)-N-{5-[amino(imino)methyl]-2-thienyl}methyl-1-{(2R)-2-[(carboxymethyl)amino]-3,3-diphenylpropanoyl}-2-pyrrolidinecarboxamide (hereinafter referred to as “Drug A”) could be orally absorbed well on an empty stomach, but its bioavailability was remarkably decreased when administered after food intake. Other oral thrombin inhibitors under development than Drug A have been reported to show changes of bioavailability by food intake. For example, the bioavailability of a thrombin inhibitor known as S-18326 was 27% on an empty stomach but decreased to 6% after food intake when administered to dogs. Another thrombin inhibitor, S-31922, has 36% and 22% of bioavailability on an empty stomach and after food intake, respectively, in dogs, and therefore, it was shown to have much less effect by food intake than S-18326 (Vallez M-O, Different food interaction for the orally active thrombin inhibitors S18326 and S31922 in dogs, XVIIth Congress of the International Society for Thrombosis and Haemostasis, Washington D.C., U.S.A., Poster 2289). The above two substances have different activities not only on thrombin but also on trypsin. That is, IC₅₀ values of S18326 and S13922 on thrombin are 3.6 nM and 43 nM, respectively, and those on trypsin are 20 nM and 340 nM, respectively. In short, S31922 with relatively low activity on trypsin showed less decreased bioavailability than S18326 by food intake. In other words, the bioavailability of S18326 with higher activity on trypsin was more affected by food intake than S31922. Another oral thrombin inhibitor, R-Piq-Pro-Arg-H, is orally absorbed well, but its bioavailability is remarkably decreased after food intake in rats and humans (R. T. Shuman and P. D. Gesellchen, 1998, Development of an orally active tripeptide arginal thrombin inhibitor, in: Pharmaceutical Biotechnology Vol 11. Integration of Pharmaceutical Discovery and Development, p57-80, Plenum, New York). Its activity on trypsin has not yet been reported, but since it is an ariginine (Arg) derivative like S18326, it is anticipated to have high activity on trypsin. An orally active thrombin inhibitor, melagatran, was reported to show remarkably decreased bioavailability by food intake in humans. The bioavailability of melagatran is affected by food intake because it has a charge in the intestinal pH, and so its membrane permeability is decreased [D. Gustafsson et al., The direct thrombin inhibitor melagatran and its oral prodrug H376/95: Intestinal absorption properties, biochemical and pharmacodynamic effects, Thrombosis Res., 101(2001) 171-181]. In order to improve the decreased membrane permeability, its prodrug was prepared and confirmed to have improved bioavailability and to prevent the bioavailability variance by food intake. However, melagatran is also one in the series of strong basic group, amidine, and thus has a strong inhibitory effect on trypsin.

In conclusion, many thrombin inhibitors that have been known heretofore often show activities on various serine proteases, particularly, trypsin, in vivo. However, it would be preferable to remove their activities on trypsin for selective inhibition of thrombin.

### DISCLOSURE OF THE INVENTION

The present inventors conceived that the decrease in bioavailability of a thrombin inhibitor active on trypsin by food intake could be improved by minimizing the interaction of the drug with trypsin in the intestinal tract after oral administration, and conducted extensive studies thereon. As a result, the present inventors developed orally administrable pharmaceutical compositions and methods which can improve the decrease in bioavailability of the drugs by food intake, and thus, completed the present invention.

Therefore, a purpose of the present invention is to provide orally administrable pharmaceutical compositions for improving the decrease of bioavailability of a drug by food intake. Another purpose of the present invention is to provide methods for improving the decrease of bioavailability of a drug by food intake.

The present invention is applicable not only to thrombin inhibitors but also to other peptidomimetic drugs active on trypsin, and other drugs active on other digestive enzymes than trypsin. Also, the present invention is applicable for improving the food effect by preventing the interaction of the drug with food ingredients in case the bioavailability of a drug is decreased by binding with specific ingredients of food as well as digestive enzymes.

One aspect of the present invention relates to an orally administrable pharmaceutical composition, for example, in the form of granule or pellet, for preventing the decrease in bioavailability of a drug by food intake, wherein said drug's bioavailability is decreased by interaction with digestive enzymes or food ingredients after food intake, comprising:

i) said drug; and

ii) a pharmaceutically acceptable bioadhesive polymer.

The above composition may further comprise one or more pharmaceutically acceptable additives, for example, solubilizing agent, osmotic agent, disintegrator, lubricant, binder, filler, and the like. Further, the composition may have an enteric coating, or a film coating applied onto the enteric coating.

Another aspect of the present invention relates to an orally administrable formulation, for example, in the form of soft or hard capsule or tablet, prepared from the above composition. The formulation may be prepared by adding one or more pharmaceutically acceptable additives, for example, solubilizing agent, osmotic agent, disintegrator, lubricant, binder, filler, and the like, to the above composition.

A third aspect of the present invention relates to a method for preventing the decrease in bioavailability of a drug by food intake, which comprises using the above composition.

Hereinafter, the present invention will be explained in detail.

The present invention provides an orally administrable pharmaceutical composition, for example, in the form of granule or pellet, comprising a drug whose bioavailability is affected by food intake, and a pharmaceutically acceptable bioadhesive polymer. Also, the present invention provides an orally administrable formulation, for example, in the form of capsule or tablet, prepared from the above composition. The composition and the formulation according to the present invention are applicable for various drugs whose bioavailability is affected by food intake, and comprise a bioadhesive polymer, and if desired, one or more pharmaceutically acceptable additives such as solubilizing agent, osmotic agent, disintegrator, lubricant, binder, filler, and the like. Furthermore, if desired, the composition of the present invention may be enteric-coated, or enteric-coated and film-coated to improve the drug release profile in the gastrointestinal tract. Also, the contents of the composition may be suitably adjusted depending on a drug's solubility according to the pH, dosage, and so on.

A wide variety of drugs whose bioavailability is affected by food intake are known. However, the present inventors first disclosed in the U.S. Ser. No. 10/280,587 those drugs whose bioavailability is affected due to interaction with the digestive enzymes. The examples of drugs active on digestive enzymes, to which the present invention is applicable, include the compounds of the following formula:

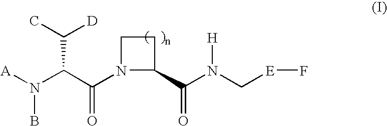

, wherein n is 1, 2, or 3;

A is hydrogen, alkyl, C₃₋₇cycloalkyl, aryl, —SO₂R¹, —SO₃R¹, —COR¹, —CO₂R², PO(OR¹)₂, —(CH₂)ₘCO₂R¹, —(CH₂)ₘSO₂R¹, —(CH₂)ₘSO₃R¹, or —(CH₂)ₘPO(OR¹)₂,

wherein R¹ is hydrogen, C₁₋₆alkyl, C₃₋₇cycloalkyl, aryl, —(CH₂)ₘaryl, or —NR³R⁴,

R² is C₁₋₆alkyl, C₃₋₇cycloalkyl, aryl, —(CH₂)ₘaryl, or alkenyl,

m is 1, 2, or 3,

wherein aryl is unsubsituted or substituted phenyl or 5-6 membered aromatic heterocyclic ring,

R³ and R⁴ are independently of each other hydrogen, C₁₋₆alkyl, or C₃₋₇cycloalkyl;

B is hydrogen or C₁₋₆alkyl;

C and D are independently of each other hydrogen, unsubsituted or substituted phenyl with one or two substituents selected from C₁₋₄alkyl, C₁₋₄alkoxy, CF₃, methylenedioxy, halogen, hydroxy, —NR³R⁴, C₃₋₇cycloalkyl, or a 5-6 membered heterocyclic ring system which may be saturated or unsaturated and consists of carbon atoms and 1-3 heteroatoms selected from the group consisting of N, O, and S;

E is

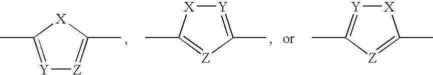

, wherein X is S, O, or NR⁵,

Y and Z are independently of each other N or CR⁶,

wherein R⁵ is hydrogen or C₁₋₄alkyl, and R⁶ is hydrogen, halogen, CF₃, or C₁₋₄alkyl; and

F is —C(NH)N(R⁷)₂, —C(NH₂)NN(R⁷)₂, —C(NH₂)NOH, or —CH₂NH(R⁷)₂,

wherein R⁷ is same or different, and is hydrogen, C₁₋₄perfluoroalkyl, or C₁₋₄alkyl, or a pharmaceutically acceptable salt thereof, as set forth in WO 00/39124, the whole contents of which are incorporated hereinto by reference.

A particularly preferable example of the compounds of formula (I) is Drug A as above described, whose chemical structure is as follows:

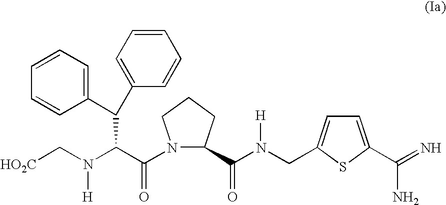

Drug A is an orally active thrombin inhibitor, and is effective for preventing or treating the formation of thrombus. Therefore, the above drug may be used for preventing or treating thrombosis, other cardiovascular disorders such as myocardial infarction, unstable angina, deep vein thrombosis, and pulmonary thrombosis, stroke, or other disorders associated with excessive thrombin. This drug also has high activity on the digestive enzyme, trypsin. When a solution of Drug A is administered to dogs, its bloavailability was reduced to 10% after food intake compared with when on an empty stomach. A key for successful application of this drug is to overcome such serious food effect by designing an effective oral formulation.

Therefore, the present invention provides an orally administrable pharmaceutical composition comprising a thrombin inhibitor, for example, a compound of formula (I) or a pharmaceutically acceptable salt thereof, particularly, Drug A, and one or more pharmaceutically acceptable bioadhesive polymers, and optionally, one or more pharmaceutically acceptable additives. The composition may comprise a solubilizing agent, an osmotic agent, a disintegrator, a lubricant, a binder, a filler, and the like. The present invention also provides an orally administrable formulation, e.g. in the form of capsule or tablet, prepared from the above composition. The formulation of the present invention may be prepared by adding a solubilizing agent, an osmotic agent, a disintegrator, a lubricant, a binder, a filler, etc. to the above composition.

Further, the composition of the present invention may be enteric-coated. The enteric coating may maximize the effects of the bioadhesive formulation of the present invention. That is, the enteric coating on the above composition may suppress the drug dissolution in the stomach to make the drug be released in the small intestine which is the absorption site of the drug. If a drug is released in the stomach and enters the small intestine in the solution state, the drug can interact with trypsin, and so its absorption is prevented. However, the enteric coating may suppress the drug release in the stomach to make the entire formulation arrive at the small intestine and adhere to the intestinal membrane, and therefore, make the drug maximally available for absorption at the major absorption site. On the other hand, the composition containing an osmotic agent, a disintegrator, etc. promotes the drug release in the small intestine where the osmotic pressure is increased due to food intake to improve the absorption of the drug. Also, in order to more preferably control the drug release in the gastrointestinal tract, an osmotic agent or disintegrator may be added to the composition before the enteric coating, or film-coating, may be ftirther applied after the enteric coating to more preferably control the dissolution of the enteric coating.

Other examples of active compounds on digestive enzymes to which the present invention is applicable include, but are not limited to, S-18326, S-31922, R-Piq-Pro-Arg-H, and melagatran.

In the present invention, the examples of pharmaceutically acceptable bioadhesive polymer include polyethylene oxide, cellulose ether, polyvinyl pyrrolidone (PVP), acrylic acid polymer, mucin, agar, gelatin, pectin, alginate, and other natural gum. Preferably, they include Polyox (polyethylene oxide, Dow Chemical), Metolose (hydroxypropyl methylcellulose (HPMC), ShinEtsu), Carbopol (BFGoodrich), and their mixtures. These polymers are hydrated thereby increasing the viscosity to have the adhesive property to the mucous membrane, and their mucoadhesiveness has been reported in the catalogues of manufacturers or prior arts. For example, EP 0 514 008 A1 showed the mucoadhesiveness of the above polymers by showing that the particles consisted of these polymers were well adhered to the intestinal membrane of rats. The above application increased the bioavailability of a drug by granule or coating containing polyglycerol fatty acid ester or lipid in combination with the drug and bioadhesive polymer. However, differently from the above application, the present invention revealed that the decrease of bioavailability of a drug by food intake may be improved by preparing granule or pellet containing the drug and a bioadhesive polymer, or an oral formulation containing the above granule or pellet and optionally various pharmaceutically acceptable additives such as solubilizing agent, osmotic agent, disintegrator, lubricant, binder, filler, and the like.

Polyox (Dow Chemical) which can be used in the present invention is a water-soluble polymer, polyethylene oxide, and has different viscosity and bioadhesiveness in an aqueous solution depending on its average molecular weight (for example: WSR 301: average MW 4,000,000, viscosity of 1% solution 1650-5500 cP; WSR N-12K: average MW 1,000,000, viscosity of 1% solution 400-800 cP; WSR N-750: average MW 300,000, viscosity of 5% solution 600-1200 cP; WSR N-10: average MW 100,000, viscosity of 5% solution 30-50 cP). Polyox may be granulated using a high shear granulator, melt extrusion, or a roller compactor.

Carbopol (BFGoodrich) which can be used in the present invention is a resin wherein an acrylic acid polymer is chemically cross-linked with polyalkenyl alcohol and divinyl glycol, and Carbopol 934P NF, 974P NF, 971P NF, etc. are used for oral use. Theses resins form highly viscous gel and are swelled upon contacting with water. Carbopol-containing granules may be prepared by a dry method by roller compaction, a wet method using water or alcohol as the binding solution, or other methods by extrusion.

The examples of cellulose ether which can be used in the present invention include hydroxypropyl methylcellulose, hydroxyethyl cellulose, and the like. Cellulose ether-containing granules may be prepared by a dry method by roller compaction, a wet method by water or alcohol as a binding solution, other methods by extrusion.

In the present invention, a pharmaceutically acceptable solubilizing agent may be contained in granules or pellets, or may not be contained in granules or pellets but may be added during the preparation process of capsules or tablets. In case of basic drug, the solubility in water is increased as the pH is decreased, and therefore, the examples of solubilizing agent which can be used in the present invention include pharmaceutically acceptable citric acid, tartaric acid, fumaric acid, maleic acid, malic acid, etc. Other examples of pharmaceutically acceptable solubilizing agent are natural surfactants such as lecithin, glycerophospholipid, sphingophospholipid, sucrose, aliphatic acid ester, bile salt, etc., surfactants such as sorbitan aliphatic acid ester (sorbitan monolaurate, sorbitan monooleate, sorbitan monostearate, etc.), polyoxyethylene sorbitan aliphatic acid ester [polyoxyethylene 20 sorbitan monolaurate (Tween 20), polyoxyethylene 20 sorbitan monooleate (Tween 80), polyoxyethylene sorbitan monostearate, polyoxyethylene sorbitan monopalmitate, etc.], polyoxyethylene castor oil derivative [polyoxyl 40 hydrogenated castor oil (Cremophor RH40, BASF), polyoxyl 35 castor oil (Cremophor EL, BASF), polyoxyl 60 hydrogenated castor oil (Cremophor RH60, BASF), etc.], polyoxyethylene glycerol oxystearate, poloxamer, etc., and their mixtures. Such solubilizing agents may or may not be necessary depending on the drug's physical properties such as solubility, etc.

The examples of pharmaceutically acceptable lubricant which can be used in the present invention include dicalcium phosphate, talc, fumed silica, stearic acid, magnesium stearate, sodium glycofumarate, etc. Such lubricants may be contained in the granules depending upon granulating methods, or may be added during the preparation process of pellet, or capsule or tablet.

In the present invention, an enteric polymer refers to any polymer which is not dissolved at the stomach' pH, but dissolved at the intestine's pH. The specific examples of this polymer include hydroxypropyl methylcellulose phthalate, cellulose acetate phthalate, carboxymethyl ethylcellulose (CMEC AQ, Kohjin Co., Ltd. Japan), and methacrylic acid methyl methacrylate copolymer (Eudragit L100-55, L100 and S100, Rohm Pharma GmbH, Germany). Such enteric polymer may be used alone or in combination. Also, ACRYL-EZE™ (Colorcon), which is prepared by combining an enteric polymer with other necessary additives for preparing a coating solution and is dispersed in water to be directly used for the enteric coating, may be used in the present invention.

The osmotic agent in the present invention refers to any water-soluble additive which plays a role to promote the absorption of water from the small intestinal juice to the above composition by the difference of osmotic pressures when contained in or coated on the composition, and thus to induce the rapid release of a drug. The examples of the water-soluble additives which can be used as the osmotic agent include saccharides such as lactose, sucrose, mannitol, dextrose, fructose, etc., and their mixtures, organic acids such as tartaric acid, citric acid, fumaric acid, maleic acid, malic acid, etc., and salts such as sodium chloride, potassium chloride, sodium phosphate, etc. A different series of osmotic agents may be used in combination depending on the desired osmotic pressure. In the present invention, the composition may contain, or may be coated with, a disintegrator to increase the drug release rate in the small intestine. A combination of osmotic agent and disintegrator may be used to obtain a more preferable effect. Herein, the examples of disintegrator include conventionally used ones, and a film forming polymeric disintegrator is preferably used when the composition is coated with disintegrator or with the combination of disintegrator and osmotic agent. The examples of disintegrator include alginic acid, calcium carboxymethylcellulose, microcrystalline cellulose (e.g. avicel), polacrillin potassium (e.g. Amberlite), sodium alginate, sodium starch-glycollate, starch, etc.

In the present invention, a film coating may be further performed on the enteric coating to more preferably control the dissolution of the enteric coating. For this purpose, various coating polymers or suitable mixtures thereof may be used. For example, coating polymers such as hydroxypropyl methylcellulose, hydroxyethyl cellulose, hydroxypropyl cellulose, methylcellulose, ethylcellulose, acrylic acid methacrylic acid ester copolymer such as Eudragit RL, Eudragit RS, etc., or a suitable ratio of mixture thereof may be used.

The composition of the present invention may contain 1 to 90 parts by weight, 10 to 60 parts by weight, preferably, of a drug whose bioavailability is decreased by interaction with digestive enzymes, particularly, trypsin, or food ingredients after food intake, and 10 to 99 parts by weight, preferably, 10 to 95 parts by weight, of a pharmaceutically acceptable bioadhesive polymer. If any, the pharmaceutically acceptable solubilizing agent may be contained at 1 to 1000 parts by weight, preferably 100 to 500 parts by weight, more preferably 30 to 100 parts by weight, in the composition. If any, the disintegrator may be contained at 1 to 1000 parts by weight, preferably 10 to 500 parts by weight, more preferably 30 to 100 parts by weight, in the composition.

The granule of the present invention may be prepared according to the wet method using a high speed granulator, the dry method using a roller compactor, slug, etc., melt extrusion, melt aggregation, or melt spheronization. Alternatively, it may be prepared by coating the powder or granule containing the thrombin inhibitor with the bioadhesive polymer through using a flow coater, etc. The granule according to the present invention preferably has the particle size distribution where 95% or more of the particles are 2 mm or less, and more preferably, 95% or more of the particles are 1 mm or less.

To prepare the pellet of the present invention, a mixed powder of drug and bioadhesive polymer, or a mixed powder further containing other pharmaceutically acceptable additives, e.g. solubilizing agent, osmotic agent, disintegrator, lubricant, binder, filler and their suitable mixtures may be directly manufactured into tablet in a tabletting machine. Also, said granule, i.e. prepared by the wet method, the dry method using a roller compactor, slug, etc., melt extrusion, melt aggregation, melt spheronization, etc., may be manufactured into tablet in a tabletting machine. In the present invention, the pellet preferably has a diameter of 4 mm or less, more preferably 3.5 mm or less, much more preferably 3 mm or less, to prevent the delay of its transfer from the stomach to the small intestine.

In the present invention, the granule may be coated in a flow coater or a centrifugal flow coater, and the pellet may be coated in a flow coater, a pan coater, a centrifugal flow coater, and so on.

### BEST MODE FOR CARRYING OUT THE INVENTION

The present invention will be more specifically illustrated by the following examples. However, the following examples should not be construed as limiting the scope of the present invention in any way.

### EXAMPLE 1

Drug A, Polyox 301, and magnesium stearate (lubricant) were mixed at the weight ratio of 5:5:0.1, and then, the mixture was tabletted in a single punch press. The obtained tablets were ground in a mortar, and sieved to obtain granules with a particle size of 0.3 to 1 mM. The granules containing 100 mg of Drug A were filled into gelatin capsules to prepare capsules.

### EXAMPLE 2

Capsules were prepared according to substantially the same method as Example 1 except that Drug A, Polyox 301, and magnesium stearate (lubricant) were mixed at the weight ratio of 6:4:0.1.

### EXAMPLE 3

Drug A and Polyox 301 were mixed at the weight ratio of 5:5, and then, the mixture was granulated in a high shear granulator while atomizing water. At this time, the supplied amount of water was 20% or less of the total weight of the mixture. The obtained granules were dried and sieved to obtain granules with a particle size of 0.3 to 2 mm. The granules containing 100 mg of Drug A were filled into gelatin capsules to prepare capsules.

### EXAMPLE 4

Capsules were prepared according to substantially the same method as Example 3 except that Drug A and Polyox 301 were mixed at the weight ratio of 4:6.

### EXAMPLE 5

Drug A and Polyox 301 were mixed at the weight ratio of 5:5, and the mixture was granulated by a dry method in a roller compactor. At this time, the operating condition was as follows: roll pressure: 5 ton; side seal pressure: 0.3 ton; roll speed: 10 rpm; screw speed: 20 rpm; and granule sieve: #18. The granules with a particle size of 1 mm or less passing through sieve # 18 were filled into gelatin capsules to prepare capsules.

### EXAMPLE 6

Capsules were prepared according to substantially the same method as Example 1 except that Drug A, HPMC (Metolose 60SH, 4000 cp), and magnesium stearate were mixed at the weight ratio of 5:5:0.1.

### EXAMPLE 7

Capsules were prepared according to substantially the same method as Example 1 except that Drug A, Carbopol 934NF, PVP K30, and magnesium stearate were mixed at the weight ratio of 4:4:2:0.1.

### EXAMPLE 8

Each 250 g of Drug A and HPMC (Metolose 60SH, 4000 cp) were introduced into a plastic bag, and shaken to mix for 5 minutes to prepare dry granules in a roller compactor. The operating condition was as follows: roll pressure: 5 ton; side seal pressure: 0.3 ton; roll speed: 10 rpm; screw speed: 20 rpm; and granule sieve: #18. As a result, 450 g of granules were obtained, and 10 g of fine powder was recovered. The granules with a size of 0.355 to 1 mm among the granules were 235 mg, and the granules with the size of 0.355 mm were 190 g. The granules containing 100 mg of Drug A were filled into gelatin capsules to prepare capsules.

### EXAMPLE 9

Drug A and Polyox 301 were mixed at the weight ratio of 5:5, and the mixture was granulated in a roller compactor. Then, the granules with a size of 0.3 to 1 mm and 480 mg of tartaric acid were filled into gelatin capsules to contain Drug A of 10 mg.

### EXAMPLE 10

A maleate salt form of Drug A, Polyox 301, and magnesium stearate were mixed at the weight ratio of 8:12:0.2, and then, the mixture was tabletted. Then, the obtained tablets were ground in a mortar, and passed through a standard mesh sieve to obtain granUles passing through #18 but not through #35. The obtained granules were filled into gelatin capsules to contain Drug A of 10 mg.

### EXAMPLE 11

A maleate salt form of Drug A, Polyox 301, and Polyox 205 were mixed at the weight ratio of 2:1:1, 1% magnesium stearate was added thereto, and the whole mixture was well mixed. A medium chain triglyceride, Miglyol, of 1 ml was added to the obtained mixture of 5 g, 3 ml of distilled water was added thereto, and the whole mixture was kneaded. Spherical pills with a diameter of 2.5 mm or less were prepared and air dried at room temperature for 2 hours. The dried pills were coated with a 20% (w/w) coating solution of ACRYL-EZE™ in a flow coater (FREUND, Japan, Model FL-Mini). The operating condition of the flow coater was as follows: inlet temperature: 42° C.; outlet temperature: 37-38° C.; spray (on 0.4/off 0.1 min); pulse jet (on 4/off 1 sec); spray pressure: 0.2 mPa; air volume flow: setting 60-62; and feeding rate of the coating solution (pump, MP-3): 1.4 g/min. The enteric coated pills were filled into gelatin capsules to contain 20 mg of Drug A.

### EXAMPLE 12

A maleate salt form of Drug A, Polyox 301, and Polyox 205 were mixed at the weight ratio of 2:1:1, 4% explotab and 1% magnesium stearate were added thereto, and the whole mixture was well mixed and manufactured into tablets in a tabletting machine. The obtained tablets were introduced into a mortar and were ground to prepare granules. The obtained granules were sieved through #18 and #35, to select ones passing through #18 but not through #35. On the other hand, Avicel PH101 was manufactured into tablets in a tabletting machine. The obtained tablets were introduced into a mortar and ground to obtain granules. The obtained granules were sieved through #18 and #35, to select ones passing through #18 but not through #35. The granules containing the maleate salt form of Drug A and the granules of Avicel PH101 were mixed at the weight ratio of 2:3, and the mixture was manufactured into tablets to obtain pellets with the diameter of 3.5 mm and the height of 3.0 mm.

The obtained pellets were coated with a 20% (w/w) coating solution of ACRYL-EZE™ in a flow coater (FREUND, Japan, Model FL-Mini). The operating condition of the flow coater was as follows: inlet temperature: 42° C.; outlet temperature: 35° C.; spray (on 0.4/off 0.1 min); pulse jet (on 4/off 1 sec); spray pressure: 0.2 mPa; air volume flow: setting 60-62; and feeding rate of the coating solution (pump, MP-3): 1.4 g/min. The enteric coated pellets were filled into gelatin capsules to contain 14 mg of Drug A.

### EXAMPLE 13

A maleate salt form of Drug A, HPMC (60SH, 4000 cp), and Polyox 301 were mixed at the weight ratio of 5:2:3, 1% magnesium stearate was added thereto, and the whole mixture was well mixed and manufactured into granules in a roller compactor. The obtained granules were sieved through #18 and #35, to select ones passing through #18 but not through #35. On the other hand, starch was manufactured into tablets in a tabletting machine. The obtained tablets were introduced into a mortar and ground to obtain granules. The obtained granules were sieved through #18 and #35, to select ones passing through #18 but not through #35. Further, Avicel PH101 was manufactured into tablets in a tabletting machine. The obtained tablets were introduced into a mortar and ground to obtain granules. The obtained granules were sieved through #18 and #35, to select ones passing through #18 but not through #35. The above three kinds of granules were mixed at the weight ratio of 3:3:1, and the mixture was manufactured into tablets to obtain pellets with the diameter of 3.5 mm and the height of 3.0 mm.

The obtained pellets were coated with a 20% (w/w) coating solution of ACRYL-EZE™ in a flow coater (FREUND, Japan, Model FL-Mini). The operating condition of the flow coater was as follows: inlet temperature: 42° C.; outlet temperature: 35° C.; spray (on 0.4/off 0.1 min); pulse jet (on 4/off 1 sec); spray pressure: 0.2 mPa; air volume flow: setting 60-62; and feeding rate of the coating solution (pump, MP-3). 1.4 g/min. The enteric coated pellets were filled into gelatin capsules to contain 23 mg of Drug A.

### EXAMPLE 14

A maleate salt form of Drug A, Polyox 301, and Polyox 205 were mixed at the weight ratio of 5:2.5:2.5, 1% magnesium stearate was added thereto, and the whole mixture was well mixed and manufactured into granules in a roller compactor. The obtained granules were sieved through #18 and #35, to select ones passing through #18 but not through #35. Avicel was manufactured into granules in a roller compactor, and the obtained granules were sieved through #18 and #35, to select ones passing through #18 but not through #35. Also, lactose was manufactured into granules in a roller compactor, and the obtained granules were sieved through #18 and #35, to select ones passing through #18 but not through #35. The above three kinds of granules were mixed at the weight ratio of 4:3:3, and the mixture was manufactured into pellets with the diameter of 3.5 mm.

The obtained pellets were coated with a 20% (w/w) coating solution of ACRYL-EZE™ in a flow coater (FREUND, Japan, Model FL-Mini). The operating condition of the flow coater was as follows: inlet temperature: 55° C.; outlet temperature: 30-32° C.; spray pressure: 0.1 mPa; feeding rate of the coating solution: 0.8 ml/min; and pan speed: 12 rpm.

### EXAMPLE 15

The enteric coated pellets obtained from Example 14 were film coated with Eudragit RL/RS (3:1). The film-coating condition was as follows: inlet temperature: 85° C.; outlet temperature: 36-38° C.; spray pressure: 0.1 mPa; feeding rate of the coating solution: 0.8 ml/min; and pan speed: 15 rpm.

### EXAMPLE 16

The granules prepared from the mixture of Drug A and Polyox, and the granules of Avicel in Example 14 were mixed at the weight ratio of 4:6 to prepare pellets with the diameter of 3.5 mm in a tabletting machine. The obtained pellets were subcoated with HPMC, and then, sugar coated in a pan coater. The sugar coated pellets were coated with a 20% (w/w) coating solution of ACRYL-EZE™ in a flow coater (FREUND, Japan, Model FL-Mini). The coating condition was the same as in Example 12. The enteric coated pellets were film coated with Eudragit RL/RS (3:1) as in Example 15.

### COMPARATIVE EXAMPLE 1

Drug A was dissolved in glycine/HCl to the concentration of 10 mg/ml to prepare a solution.

### COMPARATIVVE EXAMPLE 2

Drug A of 100 mg was filled into gelatin capsules to prepare capsules.

### COMPARATIVE EXAMPLE 3

Drug A, lactose, and starch were introduced to a mortar at the weight ratio of 5:3:2, and kneaded with an aqueous solution of PVP-K30 while mixing them. The kneaded mixture was passed through a sieve (500 micron), and then, dried in an oven to obtain granules. The granules containing Drug A of 100 mg were filled into gelatin capsules to prepare capsules.

### EXPERIMENTAL EXAMPLE 1

### Oral Administration Test in Dogs

Beagle dogs (8-12 kg, Covance Research Product, Mich., USA) were bred in a standard experimental cage with the adjusted temperature (22±3° C.) and humidity (50±20%), and supplied with water ad libitum. For fasting condition test, dogs were fast from 18 hours before orally administering a drug. For fed condition, the prescription diet (Hill's Pet Nutrition, Kansas, U.S.A.) was provided one hour before the drug administration to dogs. The drug was orally administered with 50 ml of water. About 500 μl of the blood was withdrawn from the cephalic vein with a syringe treated with heparin, and then, centrifuged to separate plasma, and the plasma was pretreated for HPLC analysis. The blood sampling had been performed before the drug administration (control), and at 30, 60, 90, 120, 180, 240, 360, 480, and 600 minutes after the drug administration, respectively. All the plasma samples were deproteinized with 2-fold volume of methanol, and centrifuged to get a supernatant, which was analyzed with HPLC. A calibration curve was plotted within a range of 0.5 to 10 g/ml of the drug. The drug was analyzed with Shiseido Capcell-Pak C₁₈ reversed-phase column. The HPLC consisted of class-LC10A system control software, CBM-10A communication bus module, 2 LC-10AD pumps, SIL-10AXL autoinjector equipped with a sample cooler, SPD-10AV ultraviolet detector (Shimadzu, Tokyo, Japan), and GLP-2050+lazer printer (LG Electronics, Seoul, Korea). The drug was analyzed with an ultraviolet detector at the wavelength of 283 nm, and the flow rate was 1 ml/min. The mobile phase was acetonitrile of 47%, and 0.1% trifluoroacetic acid/5 mm sodium dodecyl sulfate of 53%, respectively. The retention time of the drug was about 8 minutes. All the pretreated samples were stored at −20° C., and analyzed with HPLC within 2 days. The data after oral administration were described by a graph of the drug plasma concentration versus time, and were applied to a non-compartment model using Win-Nonlin program (Scientific Consultion, N.C., USA) to calculate pharmacokinetic parameters, half-life (t_{1/2}), maximum concentration (Cₘₐₓ), maximum concentration time (Tₘₐₓ), AUC_{inf}, AUCₗₐₛₜ, and bloavailability (BA). Trapezoidal rule-extrapolation method was applied to calculate AUC, and BA was calculated using the formula (AUC_{PO} Dose_{IV})/(AUC_{IV} DOSE_{PO}).

The following Table 1 shows the bioavailability of the composition according to the present invention when Drug A was administered to dogs at the dosage of 100 mg in comparison with the bioavailability of the solution (Comparative Example 1), gelatin capsules containing only drug particles (Comparative Example 2), or gelatin capsules containing granules prepared using lactose and starch (Comparative Example 3). In all the other examples and comparative examples than Comparative Example 1 (solution), the capsules filled with 500 mg of tartaric acid were administered.

TABLE 1
Bioavailability (BA %) of each formulation in dogs

| | E. 1 | E. 2 | E. 6 | E. 7 | E. 8 | C.E. 1 | C.E. 2 | C.E. 3 |
|---|---|---|---|---|---|---|---|---|
| On an | 22 | 19 | Not | Not | 22 | 42 | 36 | Not |
| empty | | | tested | tested | | | | tested |
| stomach | | | | | | | | |
| After food | 17 | 20 | 20 | 15 | 15 | 4 | 8 | 6 |
| intake | | | | | | | | |

*E.: Example; C.E.: Comparative Example

As can be seen from the results in Table 1, the solution in Comparative Example 1 showed the highest bioavailability on an empty stomach, but its bioavailability was decreased to 4% after food intake. The capsules containing only drug particles (Comparative Example 2) also showed the decreased bioavailability after food intake. The capsules filled with the granules of lactose and starch (Comparative Example 3) showed only 6% bioavailability after food intake. By contrast, the capsules filled with the granules containing such polymers as Polyox, Metolose, Carbopol, etc. (Examples 1, 2, 6 and 7) showed 15-20% bioavailability, which was improved by 4-5 times than the solution. This may be because when the granules containing thrombin inhibitor and polymer were administered, the polymer became hydrated and viscous to adhere to the intestinal membrane, and as a result, the majority of the drug was available for absorption through the intestinal membrane without interacting with trypsin secreted by food intake. Therefore, the composition of the present invention can be applied to various drugs whose bloavailability is decreased by interaction with digestive enzymes including trypsin or specific ingredients in food.

The following Table 2 shows the bioavailability of the enteric-coated pellets (Examples 12 and 13) or pills (Example 11) in comparison with the non-enteric coated formulations (Examples 9 and 10) upon administration to dogs.

TABLE 2
Bioavailability (BA %) of each formulation in dogs

| | E. 9 | E. 10 | E. 11 | E. 12 | E. 13 |
|---|---|---|---|---|---|
| On an empty stomach | Not tested | Not tested | 8.8 | 8.9 | 7.7 |
| After food intake | 0 | 0 | 12.1 | 5.0 | 6.7 |

*E.: Example

In this test, the dosage was Drug A in a range of 10 to 23 mg per individual. This dosage was adjusted to about ⅕ compared with the above Table 1 in order to examine the absorption rate in the environment that the trypsin's effects on Drug A were increased when assuming that the secreted amount of trypsin was constant. The granules consisting of Drug A and the bioadhesive polymer (Example 9), and the granules consisting of the maleate salt form of Drug A and the bioadhesive polymer (Example 10) showed little absorption rate upon administration after food intake. By contrast, the enteric coated formulations in Examples 11, 12 and 13 showed the bioavailability of 7.7% on an empty stomach, and the remarkably high bioavailability of 6.7% compared to the formulations in Examples 9 and 10 upon administration after food intake. Higher bioavailability of enteric coated formulations than the non-enteric coated ones must be the higher fraction of drug available at the absorption site with enteric coated formulations: The drug in the enteric coated formulations is not dissolved in the stomach but enters the small intestine in the polymeric matrix, thereby avoiding the direct effect on the drug by trypsin. The bioadhesiveness of the polymer further helps more drug retains longer in the intestine enough to maintain the effective blood concentration for an extended period of time. Therefore, the enteric coating of the present invention can be applied for improving the bioavailability of various drugs whose bioavailability is reduced by interaction with other digestive enzymes or specific ingredients in food.

### Industrial Applicability

According to the present invention, the decrease in the bioavailability of a drug, particularly by interaction of the drug with digestive enzymes or food ingredients, after food intake can be effectively prevented.

What is claimed is:

1. An orally administrable pharmaceutical composition for preventing the decrease in the bioavailability of a drug after food intake, wherein the drug's bioavailability is decreased by interaction with digestive enzymes or food ingredients after food intake, comprising:
i) said drug; and
ii) a pharmaceutically acceptable bioadhesive polymer.

2. The composition of claim 1, which is in the form of granule or pellet.

3. The composition of claim 1, further comprising one or more pharmacelitically acceptable additives.

4. The composition of claim 3, wherein one or more pharmaceutically acceptable additives are selected from the group consisting of solubilizing agent, osmotic agent, disintegrator, lubricant, binder, filler, or mixture thereof.

5. The composition of claim 1, wherein the drug is a thrombin inhibitor.

6. The composition of claim 5, wherein the thrombin inhibitor is a compound of the following formula: 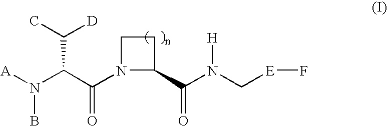
, wherein n is 1, 2, or 3;
A is hydrogen, alkyl, C₃₋₇cycloalkyl, aryl, —SO₂R¹, —SO₃R¹, —COR¹, —CO₂R², PO(OR¹)₂, —(CH₂)ₘCO₂R¹, —(CH₂)ₘSO₂R¹, —(CH₂)ₘSO₃R¹, or —(CH₂)ₘPO(OR¹)₂,
wherein R¹ is hydrogen, C₁₋₆alkyl, C₃₋₇cycloalkyl, aryl, —(CH₂)ₘaryl, or —NR³R⁴,
R² is C₁₋₆alkyl, C₃₋₇cycloalkyl, aryl, —(CH₂)ₘaryl, or alkenyl,
m is 1, 2, or 3,
wherein aryl is unsubsituted or substituted phenyl or 5-6 membered aromatic heterocyclic ring,
R³ and R⁴ are independently of each other hydrogen, C₁₋₆alkyl, C₃₋₇cycloalkyl;
B is hydrogen or C₁₋₆alkyl;
C and D are independently of each other hydrogen, unsubsituted or substituted phenyl with one or two substituents selected from C₁₋₄alkyl, C₁₋₄alkoxy, CF₃, methylenedioxy, halogen, hydroxy, or —NR³R⁴, C₃₋₇cycloalkyl, or a 5-6 membered heterocyclic ring system which may be saturated or unsaturated, and which consists of carbon atoms and 1-3 heteroatoms selected from the group consisting of N, O, and S;
E is 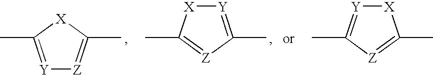
, wherein X is S, O, or NR⁵,
Y and Z are independently of each other N or CR⁶,
wherein R⁵ is hydrogen or C₁₋₄alkyl, and R⁶ is hydrogen, halogen, CF₃, or C₁₋₄alkyl; and
F is —C(NH)N(R⁷)₂, —C(NH₂)NN(R⁷)₂, —C(NH₂)NOH, or —CH₂NH(R⁷)₂,
wherein R⁷ is same or different, and is hydrogen, C₁₋₄perfluoroalkyl, C₁₋₄alkyl,
or a pharmaceutically acceptable salt thereof.

7. The composition of claim 6, wherein the compound of formula (I) is (2S)-N-{5-[amino(imino)methyl]-2-thienyl}methyl-1-{(2R)-2-[(carboxymethyl)amino]-3,3-diphenylpropanoyl}-2-pyrrolidinecarboxamine.

8. The composition of claim 5, for preventing or treating thrombosis, myocardial infarction, unstable angina, deep vein thrombosis, pulmonary thrombosis, stroke, or disorders associated with excessive thrombin.

9. The composition of claim 5, wherein the thrombin inhibitor is selected from the group consisting of S-18326, S-31922, R-Piq-Pro-Arg-H, and melagatran.

10. The composition of claim 1, wherein the bioadhesive polymer is selected from the group consisting of polyethylene oxide, cellulose ether, polyvinylpyrrolidone (PVP), acrylic acid polymer, mucin, agar, gelatin, pectin, alginate, natural gum, and their mixture.

11. The composition of claim 10, wherein the bioadhesive polymer is selected from the group consisting of Polyox, Carbopol, hydroxypropyl methylcellulose, and their mixture.

12. The composition of claim 1, which is enteric coated.

13. The composition of claim 12, which is enteric coated with hydroxypropyl methylcellulose phthalate, cellulose acetate phthalate, carboxymethyl ethylcellulose, methacrylic acid methacrylate copolymer, or mixture thereof.

14. The composition of claim 11, which is further film coated on the enteric coating.

15. The composition of claim 14, which is film coated with hydroxypropyl methylcellulose, hydroxyethyl cellulose, hydroxypropyl cellulose, methylcellulose, ethylcellulose, acrylic acid methacrylic acid ester copolymer, or mixture thereof.

16. An orally administrable formulation prepared from the composition of any one of claims 1 to 15.

17. The formulation of claim 16, which is in the form of soft or hard capsule, or tablet.

18. The formulation of claim 16, prepared by adding one or more pharmaceutically acceptable additives to the composition.

19. The formulation of claim 18, wherein one or more pharmaceutically acceptable additives are selected from the group consisting of solubilizing agent, osmotic agent, disintegrator, lubricant, binder, filler, and their mixture.

20. The formulation of claim 18, which comprises:
i) 10 to 90 parts by weight of a thrombin inhibitor;
ii) 10 to 99 parts by weight of a bioadhesive polymer; and
iii) 1 to 1000 parts by weight of a solubilizing agent, an osmotic agent, or a disintegrator.

21. A method for preventing the decrease in the bioavailability of a drug after food intake, which comprises using the composition of any one of claims 1 to 15.
